(19) United States
(12) Patent Application Publication
(10) Pub. No.: US 2009/0137023 A1
(43) Pub. Date: May 28, 2009

(54) Cytoplasm of Eukaryotic cells for reprogramming of somatic cells, healing, repairing and therapeutic applications

(75) Inventor: Brandon Casutt, Las Vegas, NV (US)

Correspondence Address: Brandon Casutt, 6829 Adobe Villa Ave., Las Vegas, NV, 89142, US

(73) Assignee: Brandon John Casutt, Las Vegas, NV (US)

(21) Appl. No.: 12/251,468

(22) Filed: Oct. 15, 2008

Related U.S. Application Data
(60) Provisional application No. 60/980,180, filed on Oct. 16, 2007.

(51) Int. Cl. C12N 7/00; C12N 5/06; C12N 5/08

(52) U.S. Cl. 435/235.1; 435/375; 435/366

(57) ABSTRACT

The present invention relates to a method for cellular reprogramming, healing and repairing for therapeutic applications by removal of the cytoplasm from the cell, collecting the cytoplasm together to form a bath of cytoplasm and then immersing one or more somatic cells into the cytoplasm bath. Alternatively, the collection of cytoplasm can be injected or mixed in with a collection of somatic cells. This is dramatically different form all other approaches were transfer of cytoplasm and/or nucleus is performed by injection from one cell directly into another cell through varies methods. This method of immersing mammalian cells into a cytoplasm environment in particular a plutipotent stem cell cytoplasm environment has many potential uses.

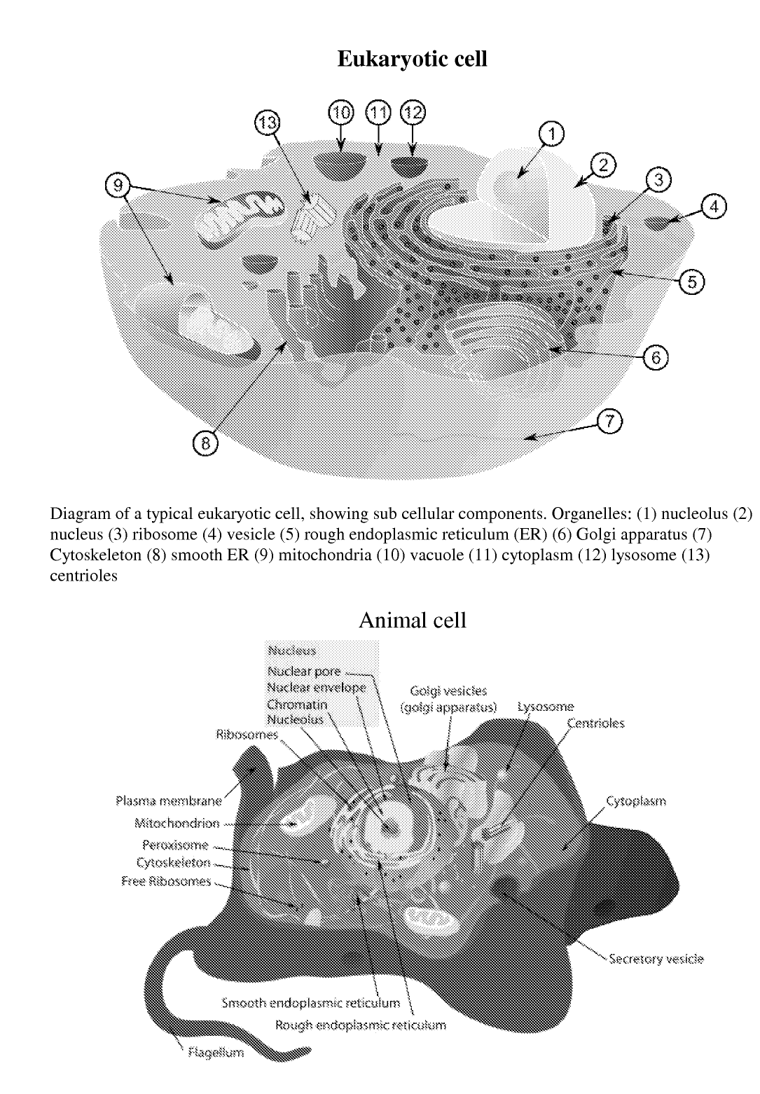

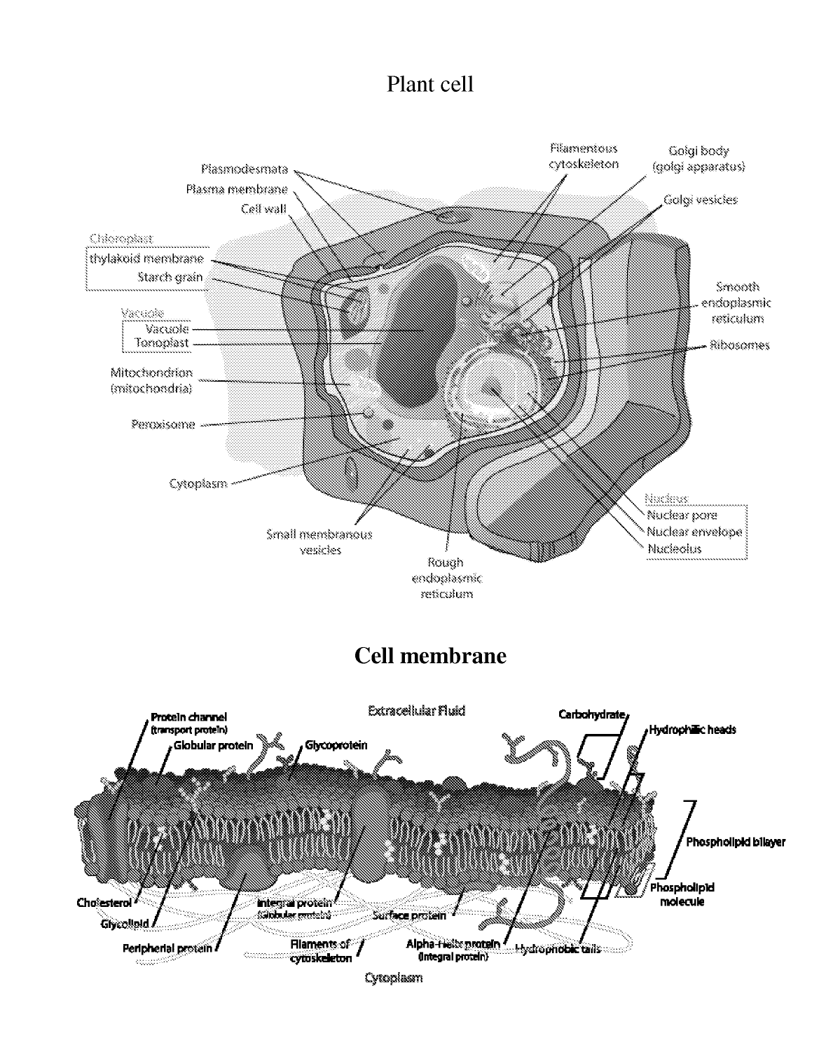

### RELATED APPLICATION INFORMATION

This nonprovisional utility patent application is claiming the benefit of a prior filed copending provisional applications. Reference to prior application No. 60/980,180, filing date 16 Oct. 2007.

### FIELD OF THE INVENTION

The present invention relates to a method for cellular reprogramming, healing and repairing for therapeutic applications by removal of the cytoplasm from the cell, collecting the cytoplasm together to form a bath of cytoplasm and then immersing one or more somatic cells into the cytoplasm bath. Alternatively, the collection of cytoplasm can be injected or mixed in with a collection of somatic cells. This is dramatically different form all other approaches were transfer of cytoplasm and/or nucleus is performed by injection from one cell directly into another cell through varies methods. This method of immersing mammalian cells into a cytoplasm environment in particular a plutipotent stem cell cytoplasm environment has many potential uses.

### BACKGROUND OF THE INVENTION

Nuclear transfer first gained acceptance in the 1960's with amphibian nuclear transplantation. (Diberardino, M. A. 1980, “Genetic stability and modulation of metazoan nuclei transplanted into eggs and ooctyes”, Differentiation, 17-17-30; Diberardino, M. A., N. J. Hoffner and L. D. Etkin, 1984; “Activation of dormant genes in specialized cells”, Science, 224:946-952; Prather, R. S. and Robl, J. M., 1991, “Cloning by nuclear transfer and splitting in laboratory and domestic animal embryos”, In: Animal Applications of Research in Mammalian Development, R. A. Pederson, A. McLaren and N. First (ed.), Cold Spring Harbor Laboratory Press.) Nuclear transfer was initially conducted in amphibians in part because of the relatively large size of the amphibian oocyte relative to that of mammals. The results of these experiments indicated to those skilled in the art that the degree of differentiation of the donor nucleus was greatly instrumental, if not determinative, as to whether a recipient oocyte containing such cell or nucleus could effectively reprogram said nucleus and produce a viable embryo. (Diberardino, M. A., N. J. Hoffner and L. D. Etkin, 1984, “Activation of dormant genes in specialized cells.”, Science, 224:946-952; Prather, R. S. and Robl, J. M., 1991, “Cloning by nuclear transfer and splitting in laboratory and domestic animal embryos”, In: Animal Applications of Research in Mammalian Development, R. A. Pederson, A. McLaren and N. First (ed.), Cold Spring Harbor Laboratory Press).

Much later, in the mid 1980s, after microsurgical techniques had been perfected, researchers investigated whether nuclear transfer could be extrapolated to mammals. The first procedures for cloning cattle were reported by Robl et al (Robl, J. M., R. Prather, F. Barnes, W. Eyestone, D. Northey, B. Gilligan and N. L. First, 1987, “Nuclear transplantation in bovine embryos”, J. Anim. Sci., 64:642-647). In fact, Dr. Robl's lab was the first to clone a rabbit by nuclear transfer using donor nuclei from earlier embryonic cells (Stice, S. L. and Robl, J. M., 1988, “Nuclear reprogramming in nuclear transplant rabbit embryos”, Biol. Reprod., 39:657-664). Also, using similar techniques, bovines (Prather, R. S., F L. Barnes, M L. Sims, Robl, J. M., W. H. Eyestone and N. L. First, 1987, “Nuclear transplantation in the bovine embryo: assessment of donor nuclei and recipient oocyte”, Biol. Reprod., 37:859-866) and sheep (Willadsen, S. M., 1986, “Nuclear transplantation in sheep embryos”, Nature, (Lond) 320:63-65), and putatively porcines (Prather, R. S., M. M. Sims and N. L. First, 1989, “Nuclear transplantation in pig embryos”, Biol. Reprod., 41:414), were cloned by the transplantation of the cell or nucleus of very early embryos into enucleated oocytes.

In the early 1990s, the possibility of producing nuclear transfer embryos with donor nuclei obtained from progressively more differentiated cells was investigated. The initial results of these experiments suggested that when an embryo progresses to the blastocyst stage (the embryonic stage where the first two distinct cell lineages appear) that the efficiency of nuclear transfer decreases dramatically (Collas, P. and J. M. Robl, 1991, “Relationship between nuclear remodeling and development in nuclear transplant rabbit embryos”, Biol. Reprod., 45:455-465). For example, it was found that trophectodermal cells (the cells that form the placenta) did not support development of the nuclear fusion to the blastocyst stage. (Collas, P. and J. M. Robl, 1991, “Relationship between nuclear remodeling and development in nuclear transplant rabbit embryos”, Biol. Reprod., 45:455-465). By contrast, inner cell mass cells (cells which form both somatic and germ line cells) were found to support a low rate of development to the blastocyst stage with some offspring obtained. (Collas P, Barnes F L, “Nuclear transplantation by microinjection of inner cell mass and granulosa cell nuclei”, Mol Reprod Devel., 1994, 38:264-267) Moreover, further work suggested that inner cell mass cells which were cultured for a short period of time could support the development to term. (Sims M, First N L, “Production of calves by transfer of nuclei from cultured inner cell mass cells”, Proc Natl Acad Sci, 1994, 91:6143-6147)

Based on these results, it was the overwhelming opinion of those skilled in the art at that time that observations made with amphibian nuclear transfer experiments would likely be observed in mammals. That is to say, it was widely regarded by researchers working in the area of cloning in the early 1990's that once a cell becomes committed to a particular somatic cell lineage that its nucleus irreversibly loses its ability to become “reprogrammed”, i.e., to support full term development when used as a nuclear donor for nuclear transfer. While the exact molecular explanation for the apparent inability of somatic cells to be effectively reprogrammed was unknown, it was hypothesized to be the result of changes in DNA methylation, histone acetylation and factors controlling transitions in chromatin structure that occur during cell differentiation. Moreover, it was believed that these cellular changes could not be reversed.

Therefore, it was quite astounding that in 1998, the Roslin Institute reported that cells committed to somatic cell lineage could support embryo development when used as nuclear transfer donors. Equally astounding, and more commercially significant, the production of transgenic cattle which were produced by nuclear transfer using transgenic fibroblast donor cells was reported shortly thereafter by scientists working at the University of Massachusetts and Advanced Cell Technology.

Also, recently two calves were reportedly produced at the Ishikawa Prefecture Livestock Research Centre in Japan from oviduct cells collected from a cow at slaughter. (Hadfield, P. and A. Coghlan, “Premature birth repeats the Dolly mixture”, New Scientist, Jul. 11, 1998) Further, Jean-Paul Renard from INRA in France reported the production of a calf using muscle cells from a fetus. (MacKenzie, D. and P. Cohen, 1998, “A French calf answers some of the questions about cloning”, New Scientist, March 21.) Also, David Wells from New Zealand reported the production of a calf using fibroblast donor cells obtained from an adult cow. (Wells, D. N., 1998, “Cloning symposium: Reprogramming Cell Fate—Transgenesis and Cloning,” Monash Medical Center, Melbourne, Australia, April 15-16)

Differentiated cells have also reportedly been successfully used as nuclear transfer donors to produce cloned mice. (Wakayama T, Perry A C F, Zucconi M, Johnsoal K R, Yanagimachi R., “Full-term development of mice from enucleated oocytes injected with cumulus cell nuclei”, Nature, 1998, 394:369-374.)

Still further, an experiment by researchers at the University of Massachusetts and Advanced Cell Technology was reported in a lead story in the New York Times, January 1999, wherein a nuclear transfer fusion embryo was produced by the insertion of an adult differentiated cell (cell obtained from the cheek of an adult human donor) into an enucleated bovine oocyte. Thus, it would appear, based on these results, that at least under some conditions differentiated cells can be reprogrammed or de-differentiated.

Related thereto, it was also reported in the popular press that cytoplasm transferred from oocyte of a young female donor “rejuvenated” an oocyte of an older woman, such that it was competent for reproduction.

However, it would be beneficial if methods could be developed for converting differentiated cells to embryonic cell types, without the need for cloning, and the production of embryos, especially given their potential for use in nuclear transfer and for producing different differentiated cell types for therapeutic use. Also, it would be beneficial if the cellular materials responsible for de-differentiation and reprogramming of differentiated cells could be identified and produced by recombinant methods, thereby improving the efficiency of cellular reprogramming.

### BRIEF SUMMARY OF THE INVENTION

The present invention relates to a method for cellular reprogramming, healing and repairing for therapeutic applications by removal of the cytoplasm from the cell, collecting the cytoplasm together to form a pool of cytoplasm and then immersing one or more somatic cells into the cytoplasm bath. Alternatively, the collection of cytoplasm can be injected or mixed in with a collection of somatic cells. This is dramatically different form all other approaches were transfer of cytoplasm and/or nucleus is performed by injection from one cell directly into another cell through varies methods. This method of immersing mammalian cells into a cytoplasm environment in particular a plutipotent stem cell cytoplasm environment has many potential uses.

### DETAILED DESCRIPTION OF THE INVENTION

Each human cell consists of a nucleus, containing its genetic material, and cytoplasm, the substance that fuels its development. Both parts of an egg contain DNA—the building blocks of life—but the nucleus contains the type of DNA that determines our physical characteristics, while the cytoplasm contains mitochondrial DNA (mtDNA) which provides energy for cellular growth. Approximately 10-15% of individuals have a similar molecular profile in their mitochondrial DNA, that occurs spontaneously.

Cytoplasm is a gelatinous, semi-transparent fluid that fills most cells. Eukaryotic cells contain a nucleus that is kept separate from the cytoplasm by a double membrane layer. The cytoplasm has three major elements; the cytosol, organelles and inclusions. The cytosol is the gooey, semi-transparent fluid in which the other cytoplasmic elements are suspended. Cytosol makes up about 80% of the cell and is composed of water, salts, organic molecules, and enzymes that are necessary for the cell to catalyze reactions. The organelles are the metabolic machinery of the cell and are like little organs themselves. The major organelles that are suspended in the cytosol consists of the mitochondria, proteins, ribosomes, the endoplasmic reticulum, the Golgi apparatus, lysosomes, and the cytoskeleton. The inclusions are chemical substances that store nutrients, secretory products and pigment granules.

Function: The cytoplasm is the site where most cellular activities are done. All the functions for cell expansion, growth and replication are carried out in the cytoplasm of the cell. The cytosol has enzymes that take molecules and break them down, so that the individual organelles can use them as they need to. The cytosol also contains the cytoskeleton which gives the cell its shape and can help in the movement of the cell.

The Cytoplasm of an Embryonic stem cell introduced around a somatic cell will cause that cell to regress into a embryonic stem cell that has the ability to proliferate and differentiate while at the same time retaining all of it's original DNA.

The cytoplasm of a cell is surrounded by a plasma membrane. Embedded within this membrane is a variety of protein molecules that act as channels and pumps that move different molecules into and out of the cell. The membrane is said to be ‘semi-permeable’, in that it can either let a substance (molecule or ion) pass through freely, pass through to a limited extent or not pass through at all. Cell surface membranes also contain receptor proteins that allow cells to detect external signaling molecules such as hormones.

A receptor is a protein on the cell membrane or within the cytoplasm or cell nucleus that binds to a specific molecule (a ligand), such as a neurotransmitter, hormone, or other substance, and initiates the cellular response to the ligand. Ligand-induced changes in the behavior of receptor proteins result in physiological changes that constitute the biological actions of the ligands.

Transmembrane receptors are integral membrane proteins, which reside and operate typically within a cell's plasma membrane, but also in the membranes of some sub cellular compartments and organelles. Binding to a signaling molecule or sometimes to a pair of such molecules on one side of the membrane, transmembrane receptors initiate a response on the other side. In this way they play a unique and important role in cellular communications and signal transduction. Many transmembrane receptors are composed of two or more protein subunits which operate collectively and may dissociate when ligands bind, fall off, or at another stage of their “activation” cycles. The polypeptide chains of the simplest are predicted to cross the lipid bilayer only once, while others cross as many as seven times (the so-called G-protein coupled receptors).

The extracellular domain is the part of the receptor that sticks out of the membrane on the outside of the cell or organelle. If the polypeptide chain of the receptor crosses the bilayer several times, the external domain can comprise several “loops” sticking out of the membrane. By definition, a receptor's main function is to recognize and respond to a specific ligand, for example, a neurotransmitter or hormone (although certain receptors respond also to changes in Transmembrane potential), and in many receptors these ligands bind to the extracellular domain.

Foreign genetic material (most commonly DNA) can also be artificially introduced into the cell by a process called transfection.

By extracting the cytoplasm from eukaryotic cells the resulting cytoplasm are then capable of binding to mammalian cells and to transfer through the cell membrane to the somatic cells inner cytoplasm it's characteristics.

By extracting the cytoplasm from an oocyte or a plutipotent stem cell the resulting cytoplasm are then capable of binding to mammalian cells and to transfer through the cell membrane to the somatic cells inner cytoplasm it's stem cell characteristics. Other unique genetic capabilities may be imparted to the somatic cells, which may serve as a vaccine for one or more pathogens or may reintroduce genetic capabilities into a mammalian host cell.

Prolonged and repeated exposure with plutipotent stem cell cytoplasm will results in the production of or reprogramming into a plutipotent stem cell.

Direct contact with plutipotent stem cell cytoplasm will results in the rejuvenating of such cells and/or restoring the proper function of the cell.

The present invention provides novel methods for de-differentiating adult somatic cells into plutipotent stem cells without generating embryos or fetuses. Cells developed using the present invention can then be differentiated into neuronal, hematopoietic, muscle, epithelial, and other cell types. These specialized cells have medical applications for treatment of degenerative diseases by “cell therapy”. These cells are desirable from a therapeutic standpoint since such cells can be used to give rise to any differentiated cell type and resultant cell types are of a genetic mach to the donor, thereby may be used in cell transplantation therapies without causing an immune response.

The present invention exploits the fact that all of the somatic cells of an individual contain the genetic information required to become any type of cell, and when placed into a conducive environment, a terminally differentiated cell's fate can be redirected to pluripotentiality. This fact has been exemplified by the success of somatic cell nuclear transfer experiments. As normal development proceeds, the gene expression profile of a cell becomes restricted and regions of the genome are stably inactivated such that, under normal conditions, the cell cannot rejuvenate. It is well-established that cell type-specific gene expression can be altered by environmental insults (as in wound healing, bone regeneration, and cancer). The present invention provides cells with extracellular and environmental clues that will induce changes in nuclear function and consequently, change the cell's identity. Using the present invention, cytoplasm from known pluripotent cell types, such as mammalian oocyte cytoplasm extract is incorporated around somatic cells. After incorporation, cells are cultured using conditions that support maintenance of de-differentiated cells (i.e. stem cell culture conditions). The dedifferentiated cells can then be expanded and induced to re-differentiate into different type of somatic cells that are needed for cell therapy; for example, into glucose responsive, insulin-producing pancreatic beta cells.

The present invention permits the memory of an adult differentiated somatic cell to be replaced with its long forgotten memory by manipulating the extra-cellular environment. By providing an adult somatic cell with factors present in an oocyte cytoplasm and/or factors present in other known pluripotent cell types, the invention restores the cells' epigenetic memory to a state of a pluripotent stem cells.

Different donor cell types are likely to require different amounts of active extract and/or different duration of delivery in order to produce the desired affect. Accordingly, different somatic cell types can be examined for their susceptibility for reprogramming, e.g. skin fibroblasts, keratinocytes, hair follicle cells, white blood cells and muscle cells. Upon demonstration that a certain cell type is particularly amenable to reprogramming, that cell type can then be used in subsequent experiments. Cell extracts obtained from different cell types are expected to display different reprogramming capacity.

The method results in the increased life-span of a mammalian cell and restoring the proper function of a somatic cell, wherein said cells or cancer cells or virus and/or bacterial infected cells.

The method results in the said cells circumventing the Hayflick limit by producing the enzyme telomerase, which regenerates telomeres during DNA replication.

The method results in individualized proper functioning cells that would then be available as a potential source of personalized, immuno-compatible regenerative therapies.

The method of claim 1, wherein the present invention provides cells with extracellular and environmental clues that will induce changes in nuclear function and consequently, change the cell's identity. said donor cell is of a plant species the same or different than the recipient cell and said donor cell's cytoplasm can comes from any Eukaryotic cell and the recipient cell can be a human somatic cell or any other eukaryotic cell of any plant or mammalian cell type.

What is claimed is:

1. A method for reprogramming and/or altering the life-span and/or strengthening of a desired cell (“recipient cell”) by introducing such cell into a cytoplasm environment from another cell type or less differentiated or undifferentiated cell (“donor cell”).

2. The method of claim 1, wherein said donor cell is an oocyte or a plutipotent stem cell.

3. The method of claim 1, wherein said cell is a mammalian cell.

4. The method of claim 3, wherein said mammalian cell is a human somatic cell.

5. The method of claim 1, which results in the production of a plutipotent stem cell.

6. The method of claim 5, which results in pluripotent stem cells, which (1) is capable of proliferating in an in vitro culture for more than one year; (2) maintains a karyotype in which the cells are euploid and are not altered through culture; (3) maintains the potential to differentiate into cell types derived from the endoderm, mesoderm and ectoderm lineages throughout the culture, and (4) is inhibited from differentiation when cultured on fibroblast feeder layers.

7. The method of claim 5, wherein pluripotent stem cells developed using the present invention can then be differentiated into neuronal, hematopoietic, muscle, epithelial, and other cell types. These specialized cells have medical applications for treatment of degenerative diseases by “cell therapy”. These cells are desirable from a therapeutic standpoint since such cells can be used to give rise to any differentiated cell type and resultant cell types are of a genetic mach to the donor, thereby may be used in cell transplantation therapies without causing an immune response.

8. The method of claim 5, wherein the present invention permits the memory of an adult differentiated somatic cell to be replaced with its long forgotten memory by manipulating the extra-cellular environment. By providing an adult somatic cell with factors present in an oocyte cytoplasm and/or factors present in other known pluripotent cell types, the invention restores the cells' epigenetic memory to a state of a pluripotent stem cells.

9. The method of claim 1, where an improved method of gene therapy that involves the introduction of at least one genetic modified gene wherein the improvement comprises using as the genetically modified cell, a mammalian cell which has been “reprogrammed” and/or having an increased life-span by the introduction of a cytoplasm environment from an oocyte or plutipotent donor stem cell of the same or different species.

10. The method of claim 1, which results in the increased heath and/or strength of a somatic cell.

11. The method of claim 1, wherein said donor cell is of a the same or different species than the recipient cell.

12. The method of claim 11, wherein said donor cell is a human or a non-human primate oocyte or either a totipotent, pluripotent, multipotent stem cell, spore-like cell and/or unipotent progenitor cell and the recipient cell is a human somatic cell.

13. The method of claim 1, which results in the increased life-span of a mammalian cell.

14. The method of claim 1, which results in restoring the proper function of a somatic cell.

15. The method of claim 14, wherein said cells or cancer cells or virus and/or bacterial infected cells.

16. The method of claim 14, wherein said cells can circumventing the Hayflick limit by producing the enzyme telomerase, which regenerates telomeres during DNA replication.

17. The method of claim 14, wherein such individualized proper functioning cells would then be available as a potential source of personalized, immuno-compatible regenerative therapies.

18. The method of claim 1, wherein the present invention provides cells with extracellular and environmental clues that will induce changes in nuclear function and consequently, change the cell's identity.

19. The method of claim 1, wherein said donor cell is of a plant species the same or different than the recipient cell.

20. The method of claim 1, wherein said donor cell's cytoplasm comes from any Eukaryotic cell and the recipient cell is a human somatic cell or any other eukaryotic cell of any plant or mammalian cell type.
